(19) United States
(12) Patent Application Publication
(10) Pub. No.: US 2005/0164290 A1
(43) Pub. Date: Jul. 28, 2005

(54) Computer software for sequence selection

(75) Inventors: Hui Wang, San Jose, CA (US); Jake Chen, San Jose, CA (US); Michael Mittmann, Palo Alto, CA (US)

Correspondence Address: AFFYMETRIX, INC;ATTN: CHIEF IP COUNSEL, LEGAL DEPT., 3380 CENTRAL EXPRESSWAY, SANTA CLARA, CA, 95051, US

(73) Assignee: Affymetrix, INC., Santa Clara, CA (US)

(21) Appl. No.: 11/084,309

(22) Filed: Mar. 18, 2005

Related U.S. Application Data
(63) Continuation of application No. 09/764,324, filed on Jan. 16, 2001.
(60) Provisional application No. 60/176,520, filed on Jan. 13, 2000.

(51) Int. Cl. C12Q 1/68; G06F 19/00; G01N 33/48; G01N 33/50

(52) U.S. Cl. 435/6; 702/20

(57) ABSTRACT

Methods, systems and computer software products are provided for nucleic acid probe array design. In one preferred embodiment, target sequences are selected from various sources and processed for probe selection.

### RELATED APPLICATIONS

This application claims the priority of U.S. Provisional Application No. 60/176,520, filed on Jan. 13, 2000. The '520 application is incorporated herein in its entireties by reference for all purposes.

This application is related to U.S. patent application Ser. No. 09/721,042, filed on Nov. 21, 2000, entitled “Methods and Computer Software Products for Predicting Nucleic Acid Hybridization Affinity”; U.S. patent application Ser. No. 09/718.295, filed on Nov. 21, 2000, entitled “Methods and Computer Software Products for Selecting Nucleic Acid Probes” and U.S. patent application Ser. No. __/______, attorney docket number 3373.1, filed on ______, entitled “Methods For Selecting Nucleic Acid Probes.” All the cited applications are incorporated herein by reference in their entireties for all purposes.

### FIELD OF INVENTION

This invention is related to bioinformatics and biological data analysis. Specifically, this invention provides methods, computer software products and systems for designing nucleic acid probe arrays.

### BACKGROUND OF THE INVENTION

The present invention relates to methods for designing nucleic acid probe arrays. U.S. Pat. No. 5,424,186 describes a pioneering technique for, among other things, forming and using high density arrays of molecules such as oligonucleotides, RNA or DNA), peptides, polysaccharides, and other materials. This patent is hereby incorporated by reference for all purposes. However, there is still great need for methods, systems and software for designing high density nucleic acid probe arrays.

### SUMMARY OF THE INVENTION

In one aspect of the invention, methods are provided for selecting sequences for designing a probe array. The methods include cleaning raw sequences; refining clusters of the raw sequences; and generating candidate design sequences, wherein the candidate design sequences are exemplar or consensus sequences of the clusters. In preferred embodiments, the cleaning process includes removing withdrawn sequences; screening and filtering and masking raw sequences; and triming terminal ambiguous sequence regions. In some embodiments, the refining includes two level clustering. The candidate design sequences may be generated by selecting exemplary sequences, preferably one for each cluster. Alternatively, the candidate design sequences may be the consensus sequence of the clusters. Exemplary methods for generating consensus sequences include generating alignments of sequences within clusters; calling consensus sequence bases according to consensus calling rules; and determining consensus sequence direction (e.g., 5′→3′ direction). When there is no contradictory direction of sequences in the cluster, the consensus sequence direction is the direction of the sequences in the cluster.

In preferred embodiments, when there are contradictory directions in a cluster, the method includes determining the probability (b) that the contradictions are explained by random errors according to a statistical model and the weighted number of contradictory sequences in the cluster; and defining the direction of majority of the sequences as the direction of the consensus sequence if the probability is the same as or greater than a threshold value (T) and x≧n/2. In preferred embodiment, the statistical model is a binomial distribution and the probability is calculated as follows:

$$b ( x ; n , p ) = \frac{n !}{x ! ( n - x ) !} {{p}^{x} ( 1 - p )}^{n - x}$$

where n is the weighted number of the sequences in the cluster; p is the probability of random errors resulting in the contradictions; and x is the number of the contradictory sequences. In some embodiments, CDS and mRNA sequences carry a higher weight than 5′ EST or 3′ EST; directionless EST carrys a weight of 0. In some other embodiments, the weights to different types of sequences are the same. The threshold value may be around 0.001, 0002 or 0.003. The methods may also include defining the direction of majority of the sequences as the direction of the consensus sequence if the probability is lower than the threshold value and x≦n*(P_{T}). In addition, the methods may include further subclustering for the minority direction and majority direction if the probability is smaller than the threshold value and x>n*p. p value may be between 0.03-0.10, preferably around 0.06. In some other preferred embodiments, the p is determined according to binomial frequency distribution of contradictory sequences in a plurality of clusters or subclusters of sequences.

The methods for resolving contradictory directions in a cluster are not only useful for sequence selection, but also for other gene indexing purposes.

In another aspect of the invention, systems and computer software are provided for sequence selection and for resolving contraductory sequence direction in clusters.

The systems include a processor; and a memory coupled with the processor, the memory storing a plurality of machine instructions that cause the processor to perform logical steps of the methods of the invention. The computer software products of the invention include a computer readable medium having computer-executable instructions for performing the methods of the invention.

### BRIEF DESCRIPTION OF THE DRAWINGS

The accompanying drawings, which are incorporated in and form a part of this specification, illustrate embodiments of the invention and, together with the description, serve to explain the principles of the invention:

FIG. 1 illustrates an example of a computer system that may be utilized to execute the software of an embodiment of the invention.

FIG. 2 illustrates a system block diagram of the computer system of FIG. 1.

FIG. 3 illustrates a computer network suitable for executing the software of an embodiment of the invention.

FIG. 4 illustrates an exemplary process for probe array design.

FIG. 5 illustrates an exemplary process for probe selection.

FIG. 6 illustrates an example assembly, shown with an exemplar sequence and a consensus sequence.

FIG. 7 illustrates a consensus sequence generated from aligned sequences.

FIG. 8 illustrates the relationships between aligned sequences in a subcluster and the consensus.

FIG. 9 illustrates a mislabeled sequence (pointed and labled as “conflicting sequence”) causes problems in determining the consensus sequence direction.

FIG. 10 shows a frequency distribution chart for sequence description contradictions from subclusters of varying size (size>=64).

### DETAILED DESCRIPTION OF THE PREFERRED EMBODIMENTS

Reference will now be made in detail to the preferred embodiments of the invention. While the invention will be described in conjunction with the preferred embodiments, it will be understood that they are not intended to limit the invention to these embodiments. On the contrary, the invention is intended to cover alternatives, modifications and equivalents, which may be included within the spirit and scope of the invention. All cited references, including patent and non-patent literature, are incorporated herein by reference in their entireties for all purposes.

### I. High Density Probe Arrays

The methods, computer software and systems of the invention are particularly useful for designing high density nucleic acid probe arrays.

High density nucleic acid probe arrays, also referred to as “DNA Microarrays,” have become a method of choice for monitoring the expression of a large number of genes and for detecting sequence variations, mutations and polymorphism. As used herein, “nucleic acids” may include any polymer or oligomer of nucleosides or nucleotides (polynucleotides or oligonucleotidies), which include pyrimidine and purine bases, preferably cytosine, thymine, and uracil, and adenine and guanine, respectively. See Albert L. Lehninger, PRINCIPLES OF BIOCHEMISTRY, at 793-800 (Worth Pub. 1982) and L. Stryer, BIOCHEMISTRY, 4^{th} Ed. (March 1995), both incorporated by reference. “Nucleic acids” may include any deoxyribonucleotide, ribonucleotide or peptide nucleic acid component, and any chemical variants thereof, such as methylated, hydroxymethylated or glucosylated forms of these bases, and the like. The polymers or oligomers may be heterogeneous or homogeneous in composition, and may be isolated from naturally-occurring sources or may be artificially or synthetically produced. In addition, the nucleic acids may be DNA or RNA, or a mixture thereof, and may exist permanently or transitionally in single-stranded or double-stranded form, including homoduplex, heteroduplex, and hybrid states.

“A target molecule” refers to a biological molecule of interest. The biological molecule of interest can be a ligand, receptor, peptide, nucleic acid (oligonucleotide or polynucleotide of RNA or DNA), or any other of the biological molecules listed in U.S. Pat. No. 5,445,934 at col. 5, line 66 to col. 7, line 51, which is incorporated herein by reference for all purposes. For example, if transcripts of genes are the interest of an experiment, the target molecules would be the transcripts. Other examples include protein fragments, small molecules, etc. “Target nucleic acid” refers to a nucleic acid (often derived from a biological sample) of interest. Frequently, a target molecule is detected using one or more probes. As used herein, a “probe” is a molecule for detecting a target molecule. It can be any of the molecules in the same classes as the target referred to above. A probe may refer to a nucleic acid, such as an oligonucleotide, capable of binding to a target nucleic acid of complementary sequence through one or more types of chemical bonds, usually through complementary base pairing, usually through hydrogen bond formation. As used herein, a probe may include natural (i.e. A, G, U, C, or T) or modified bases (7-deazaguanosine, inosine, etc.). In addition, the bases in probes may be joined by a linkage other than a phosphodiester bond, so long as the bond does not interfere with hybridization. Thus, probes may be peptide nucleic acids in which the constituent bases are joined by peptide bonds rather than phosphodiester linkages. Other examples of probes include antibodies used to detect peptides or other molecules, any ligands for detecting its binding partners. When referring to targets or probes as nucleic acids, it should be understood that these are illustrative embodiments that are not to limit the invention in any way.

In preferred embodiments, probes may be immobilized on substrates to create an array. An “array” may comprise a solid support with peptide or nucleic acid or other molecular probes attached to the support. Arrays typically comprise a plurality of different nucleic acids or peptide probes that are coupled to a surface of a substrate in different, known locations. These arrays, also described as “microarrays” or colloquially “chips” have been generally described in the art, for example, in Fodor et al., Science, 251:767-777 (1991), which is incorporated by reference for all purposes. Methods of forming high density arrays of oligonucleotides, peptides and other polymer sequences with a minimal number of synthetic steps are disclosed in, for example, U.S. Pat. Nos. 5,143,854, 5,252,743, 5,384,261, 5,405,783, 5,424,186, 5,429,807, 5,445,943, 5,510,270, 5,677,195, 5,571,639, 6,040,138, all incorporated herein by reference for all purposes. The oligonucleotide analogue array can be synthesized on a solid substrate by a variety of methods, including, but not limited to, light-directed chemical coupling, and mechanically directed coupling. See Pirrung et al., U.S. Pat. No. 5,143,854 (see also PCT Application No. WO 90/15070) and Fodor et al., PCT Publication Nos. WO 92/10092 and WO 93/09668, U.S. Pat. Nos. 5,677,195, 5,800,992 and 6,156,501, which disclose methods of forming vast arrays of peptides, oligonucleotides and other molecules using, for example, light-directed synthesis techniques. See also, Fodor, et al., Science, 251, 767-77 (1991). These procedures for synthesis of polymer arrays are now referred to as VLSIPS™ procedures.

Methods for making and using molecular probe arrays, particularly nucleic acid probe arrays are also disclosed in, for example, U.S. Pat. Nos. 5,143,854, 5,242,974, 5,252,743, 5,324,633, 5,384,261, 5,405,783, 5,409,810, 5,412,087, 5,424,186, 5,429,807, 5,445,934, 5,451,683, 5,482,867, 5,489,678, 5,491,074, 5,510,270, 5,527,681, 5,527,681, 5,541,061, 5,550,215, 5,554,501, 5,556,752, 5,556,961, 5,571,639, 5,583,211, 5,593,839, 5,599,695, 5,607,832, 5,624,711, 5,677,195, 5,744,101, 5,744,305, 5,753,788, 5,770,456, 5,770,722, 5,831,070, 5,856,101, 5,885,837, 5,889,165, 5,919,523, 5,922,591, 5,925,517, 5,658,734, 6,022,963, 6,150,147, 6,147,205, 6,153,743 and 6,140,044, all of which are incorporated by reference in their entireties for all purposes.

Microarray can be used in a variety of ways. A preferred microarray contains nucleic acids and is used to analyze nucleic acid samples. Typically, a nucleic acid sample is prepared from appropriate source and labeled with a signal moiety, such as a fluorescent label. The sample is hybridized with the array under appropriate conditions. The arrays are washed or otherwise processed to remove non-hybridized sample nucleic acids. The hybridization is then evaluated by detecting the distribution of the label on the chip. The distribution of label may be detected by scanning the arrays to determine fluorescence intensity distribution. Typically, the hybridization of each probe is reflected by several pixel intensities. The raw intensity data may be stored in a gray scale pixel intensity file. The GATC™ Consortium has specified several file formats for storing array intensity data. The final software specification is available at www.gatcconsortium.org and is incorporated herein by reference in its entirety. The pixel intensity files are usually large. For example, a GATC™ compatible image file may be approximately 50 Mb if there are about 5000 pixels on each of the horizontal and vertical axes and if a two byte integer is used for every pixel intensity. The pixels may be grouped into cells (see, GATC™ software specification). The probes in a cell are designed to have the same sequence (i.e., each cell is a probe area). A CEL file contains the statistics of a cell, e.g., the 75th percentile and standard deviation of intensities of pixels in a cell. The 50, 60, 70, 75 or 80th percentile of pixel intensity of a cell is often used as the intensity of the cell.

Nucleic acid probe arrays have found wide applications in gene expression monitoring, genotyping and mutation detection. For example, massive parallel gene expression monitoring methods using nucleic acid array technology have been developed to monitor the expression of a large number of genes (e.g., U.S. Pat. Nos. 5,871,928, 5,800,992 and 6,040,138; de Saizieu et al., 1998, Bacteria Transcript Imaging by Hybridization of total RNA to Oligonucleotide Arrays, NATURE BIOTECHNOLOGY, 16:45-48; Wodicka et al., 1997, Genome-wide Expression Monitoring in Saccharomyces cerevisiae, NATURE BIOTECHNOLOGY 15:1359-1367; Lockhart et al., 1996, Expression Monitoring by Hybridization to High Density Oligonucleotide Arrays. NATURE BIOTECHNOLOGY 14:1675-1680; Lander, 1999, Array of Hope, NATURE-GENETICS, 21(suppl.), at 3, all incorporated herein by reference for all purposes). Hybridization-based methodologies for high throughput mutational analysis using high-density oligonucleotide arrays (DNA chips) have been developed, see Hacia et al., 1996, Detection of heterozygous mutations in BRCA1 using high density oligonucleotide arrays and two-color fluorescence analysis. Nat. Genet. 14:441-447, Hacia et al., New approaches to BRCA1 mutation detection, Breast Disease 10:45-59 and Ramsey 1998, DNA chips: State-of-Art, Nat Biotechnol. 16:40-44, all incorporated herein by reference for all purposes). Oligonucleotide arrays have been used to screen for sequence variations in, for example, the CFTR gene (U.S. Pat. No. 6,027,880, Cronin et al., 1996, Cystic fibrosis mutation detection by hybridization to light-generated DNA probe arrays. Hurn. Mut. 7:244-255, both incorporated by reference in their entireties), the human immunodeficiency virus (HIV-1) reverse transcriptase and protease genes (U.S. Pat. No. 5,862,242 and Kozal et al., 1996, Extensive polymorphisms observed in HIV-1 clade B protease gene using high density oligonucleotide arrays. Nature Med. 1:735-759, both incorporated herein by reference for all purposes), the mitochondrial genome (Chee et al., 1996, Accessing genetic information with high density DNA arrays. Science 274:610-614) and the BRCA1 gene (U.S. Pat. No. 6,013,449, incorporated herein by reference for all purposes).

Methods for signal detection and processing of intensity data are additionally disclosed in, for example, U.S. Pat. Nos. 5,445,934, 547,839, 5,578,832, 5,631,734, 5,800,992, 5,856,092, 5,936,324, 5,981,956, 6,025,601, 6,090,555, 6,141,096, 6,141,096, and 5,902,723. Methods for array based assays, computer software for data analysis and applications are additionally disclosed in, e.g., U.S. Pat. Nos. 5,527,670, 5,527,676, 5,545,531, 5,622,829, 5,631,128, 5,639,423, 5,646,039, 5,650,268, 5,654,155, 5,674,742, 5,710,000, 5,733,729, 5,795,716, 5,814,450, 5,821,328, 5,824,477, 5,834,252, 5,834,758, 5,837,832, 5,843,655, 5,856,086, 5,856,104, 5,856,174, 5,858,659, 5,861,242, 5,869,244, 5,871,928, 5,874,219, 5,902,723, 5,925,525, 5,928,905, 5,935,793, 5,945,334, 5,959,098, 5,968,730, 5,968,740, 5,974,164, 5,981,174,5,981,185, 5,985,651, 6,013,440, 6,013,449, 6,020,135, 6,027,880, 6,027,894, 6,033,850, 6,033,860, 6,037,124, 6,040,138, 6,040,193, 6,043,080, 6,045,996, 6,050,719, 6,066,454, 6,083,697, 6,114,116, 6,114,122, 6,121,048, 6,124,102, 6,130,046, 6,132,580, 6,132,996 and 6,136,269, all of which are incorporated by reference in their entireties for all purposes.

Nucleic acid probe array technology, use of such arrays, analysis array based experiments, associated computer software, composition for making the array and practical applications of the nucleic acid arrays are also disclosed, for example, in the following U.S. patent applications: Ser. Nos. 07/838,607, 07/883,327, 07/978,940, 08/030,138, 08/082,937, 08/143,312, 08/327,522, 08/376,963, 08/440,742, 08/533,582, 08/643,822, 08/772,376, 09/013,596, 09/016,564, 09/019,882, 09/020,743, 09/030,028, 09/045,547, 09/060,922, 09/063,311, 09/076,575, 09/079,324, 09/086,285, 09/093,947, 09/097,675, 09/102,167, 09/102,986, 09/122,167, 09/122,169, 09/122,216, 09/122,304, 09/122,434, 09/126,645, 09/127,115, 09/132,368, 09/134,758, 09/138,958, 09/146,969, 09/148,210, 09/148,813, 09/170,847, 09/172,190, 09/174,364, 09/199,655, 09/203,677, 09/256,301, 09/285,658, 09/294,293, 09/318,775, 09/326,137, 09/326,374, 09/341,302, 09/354,935, 09/358,664, 09/373,984, 09/377,907, 09/383,986, 09/394,230, 09/396,196, 09/418,044, 09/418,946, 09/420,805, 09/428,350, 09/431,964, 09/445,734, 09/464,350, 09/475,209, 09/502,048, 09/510,643, 09/513,300, 09/516,388, 09/528,414, 09/535,142, 09/544,627, 09/620,780, 09/640,962, 09/641,081, 09/670,510, 09/685,011, and 09/693,204 and in the following Patent Cooperative Treaty (PCT) applications/publications: PCT/NL90/00081, PCT/GB91/00066, PCT/US91/08693, PCT/US91/09226, PCT/US91/09217, WO/93/10161, PCT/US92/10183, PCT/GB93/00147, PCT/US93/01152, WO/93/22680, PCT/US93/04145, PCT/US93/08015, PCT/US94/07106, PCT/US94/12305, PCT/GB95/00542, PCT/US95/07377, PCT/US95/02024, PCT/US96/05480, PCT/US96/11147, PCT/US96/14839, PCT/US96/15606, PCT/US97/01603, PCT/US97/02102, PCT/GB97/005566, PCT/US97/06535, PCT/GB97/01148, PCT/GB97/01258, PCT/US97/08319, PCT/US97/08446, PCT/US97/10365, PCT/US97/17002, PCT/US97/16738, PCT/US97/19665, PCT/US97/20313, PCT/US97/21209, PCT/US97/21782, PCT/US97/23360, PCT/US98/06414, PCT/US98/01206, PCT/GB98/00975, PCT/US98/04280, PCT/US98/04571, PCT/US98/05438, PCT/US98/05451, PCT/US98/12442, PCT/US98/12779, PCT/US98/12930, PCT/US98/13949, PCT/US98/15151, PCT/US98/15469, PCT/US98/15458, PCT/US98/15456, PCT/US98/16971, PCT/US98/16686, PCT/US99/19069, PCT/US98/18873, PCT/US98/18541, PCT/US98/19325, PCT/US98/22966, PCT/US98/26925, PCT/US98/27405 and PCT/IB99/00048, all the above cited patent applications and other references cited throughout this specification are incorporated herein by reference in their entireties for all purposes.

### III. Systems for Chip Design

In aspects of the invention, methods, computer software and systems for probe design are provided. One of skill in the art would appreciate that many computer systems are suitable for carrying out the genotyping methods of the invention. Computer software according to the embodiments of the invention can be executed in a wide variety of computer systems.

For a description of basic computer systems and computer networks, see, e.g., Introduction to Computing Systems: From Bits and Gates to C and Beyond by Yale N. Patt, Sanjay J. Patel, 1st edition (Jan. 15, 2000) McGraw Hill Text; ISBN: 0072376902; and Introduction to Client/Server Systems : A Practical Guide for Systems Professionals by Paul E. Renaud, 2nd edition (June 1996), John Wiley & Sons; ISBN: 0471133337, both are incorporated herein by reference in their entireties for all purposes.

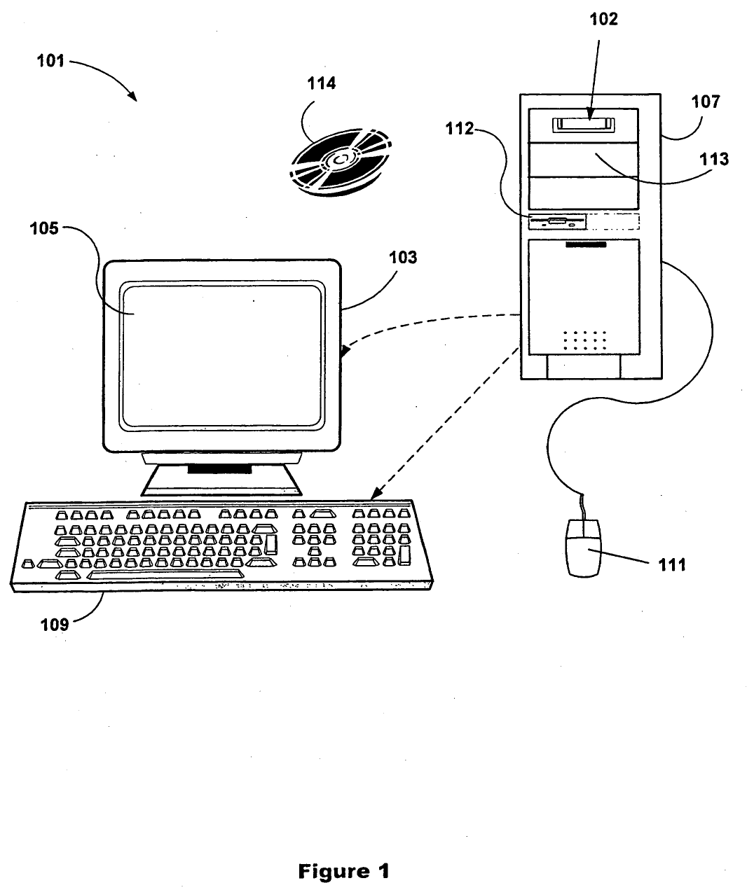

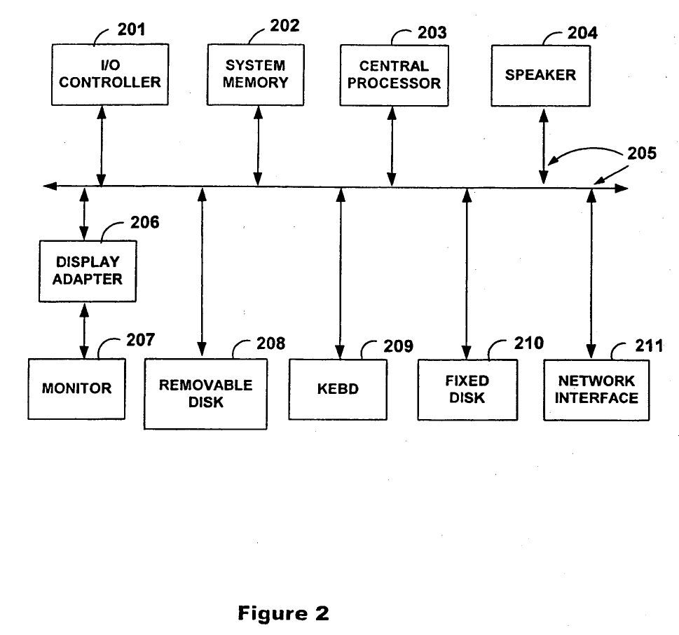

FIG. 1 illustrates an example of a computer system that may be used to execute the software of an embodiment of the invention. FIG. 1 shows a computer system 101 that includes a display 103, screen 105, cabinet 107, keyboard 109, and mouse 111. Mouse 111 may have one or more buttons for interacting with a graphic user interface. Cabinet 107 houses a floppy drive 112, CD-ROM or DVD-ROM drive 102, system memory and a hard drive (113) (see also FIG. 2) which may be utilized to store and retrieve software programs incorporating computer code that implements the invention, data for use with the invention and the like. Although a CD 114 is shown as an exemplary computer readable medium, other computer readable storage media including floppy disk, tape, flash memory, system memory, and hard drive may be utilized. Additionally, a data signal embodied in a carrier wave (e.g., in a network including the Internet) may be the computer readable storage medium.

FIG. 2 shows a system block diagram of computer system 101 used to execute the software of an embodiment of the invention. As in FIG. 1, computer system 101 includes monitor 201, and keyboard 209. Computer system 101 further includes subsystems such as a central processor 203 (such as a Pentium™ III processor from Intel), system memory 202, fixed storage 210 (e.g., hard drive), removable storage 208 (e.g., floppy or CD-ROM), display adapter 206, speakers 204, and network interface 211. Other computer systems suitable for use with the invention may include additional or fewer subsystems. For example, another computer system may include more than one processor 203 or a cache memory. Computer systems suitable for use with the invention may also be embedded in a measurement instrument.

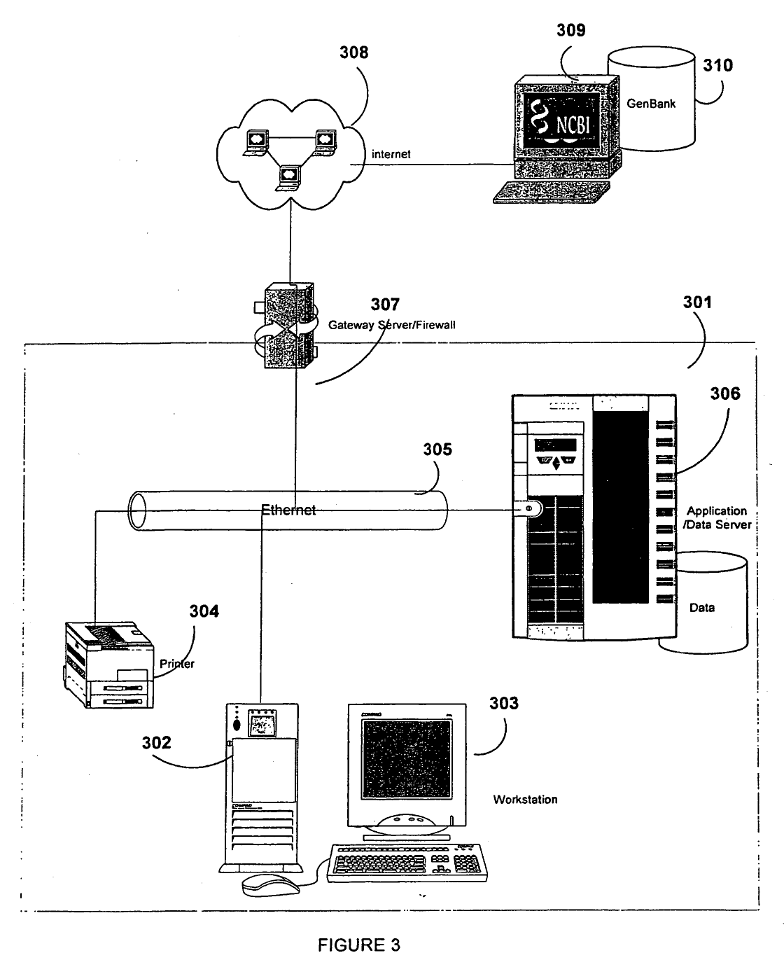

FIG. 3 shows an exemplary computer network that is suitable for executing the computer software of the invention. A computer workstation 302 is connected with the application/data server(s) through a local area network (LAN) 301, such as an Ethernet 305. A printer 304 may be connected directly to the workstation or to the Ethernet 305. The LAN may be connected to a wide area network (WAN), such as the Internet 308, via a gateway server 307 which may also serve as a firewall between the WAN 308 and the LAN 305. In preferred embodiments, the workstation may communicate with outside data sources, such as the National Biotechnology Information Center, through the Internet. Various protocols, such as FTP and HTTP, may be used for data communication between the workstation and the outside data sources. Outside genetic data sources, such as the GenBank 310, are well known to those skilled in the art. An overview of GenBank and the National Center for Biotechnology information (NCBI) can be found in the web site of NCBI (http://www.ncbi.nlm.nih.gov).

Computer software products of the invention typically include computer readable medium having computer-executable instructions for performing the logic steps of the methods of the invention. Suitable computer readable medium include floppy disk, CD-ROM/DVD/DVD-ROM, hard-disk drive, flash memory, ROM/RAM, magnetic tapes and etc. The computer executable instructions may be written in any suitable computer language or combination of several languages. Suitable computer languages include C/C++ (such as Visual C/C++), Java, Basic (such as Visual Basic), SQL, Fortran, SAS and Perl.

### IV. Sequence Selection

Methods, systems and software are provided for sequence selection. The methods, systems and software are particularly useful designing nucleic acid probe arrays, especially oligonucleotide probe arrays for gene expression monitoring. Various aspect of the invention will be described using embodiments employing gene expression probe array and UniGene database. One skilled in the art would appreciate that the methods, systems and software not limited to the specific embodiments.

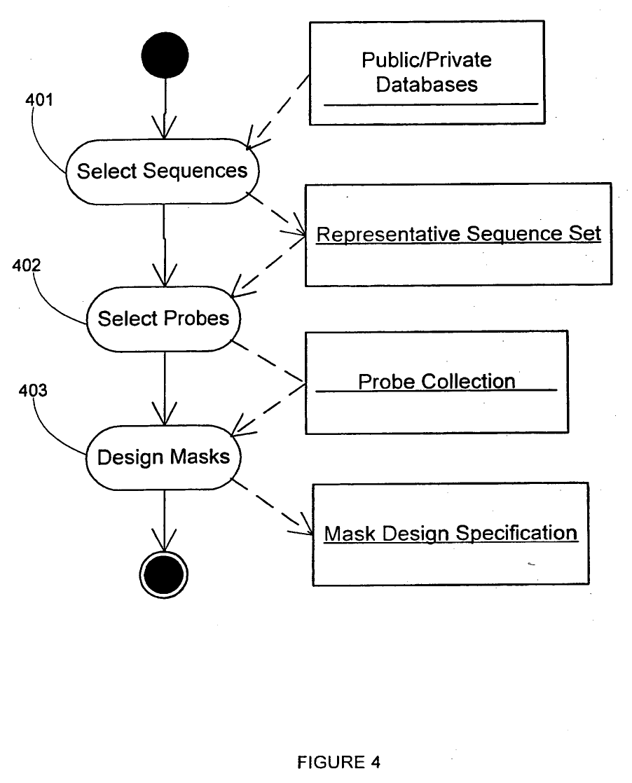

FIG. 4 shows an exemplary process for designing a gene expression probe array. Sequences from various sources are used for sequence selection 401. The source sequences may come from, e.g., genomic sequences, cDNA sequences, expressed sequence tags (ESTs) or EST clusters. The sequence selection process generates candidate sequences for probe selection 402. For photolithographic synthesis of oligonucleotide arrays, masks may be designed based upon the probe sequences 403. Processes, systems and computer software products for probe selection and mask designs are disclosed in, for example, U.S. Pat. Nos. 5,800,992, 6,040,138, 5,571,639, 5,593,839, and 5,856,101, and U.S. patent application Ser. Nos. 09/719,295, 09/721,042 and Attorney Docket Number 3273.1, all incorporated herein by reference for all purposes.

Sequence selection, as the first phase of expression chip design, is important for several reasons. First, sequence selection helps eliminate redundant sequences. Sequence redundancy is one of the main issue in public or private databases. For example, one clone can be sequenced hundreds of times (dbEST). In Genbank, different entries of mRNA, cDNA or genomic sequence can represent the same gene. Second, sequence selection helps remove low quality sequences and sequence regions. Design candidate sequences from public databases such as GenBank and UniGene generally have low quality and often contain too long or too short sequences, withdrawn sequences, ribosomal RNAs, sequences with vector contamination, repetitive elements, or low-complexity regions, sequences with too many ambiguous bases. Therefore, sequence selection preferably include stage(s) to eliminate or minimize the sources of sequence quality problems.

The sequence selection process may involve the use of clustering tools, BLAST (http://www.ncbi.nlm.nih.gov), FASTA, etc. In addition, gene identification/prediction tools, multiple alignment tools, consensus calling/assembly methods may also be employed.

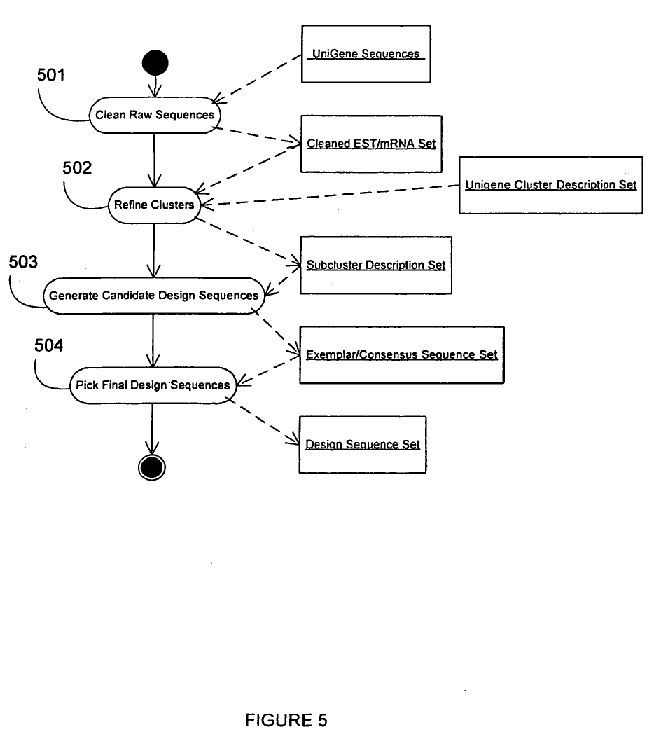

FIG. 5 shows an exemplary process for sequence selection for expression probe arrays. Raw sequence information from public or private databases, mRNA, Coding Regions (CDS), EST, gene clusters (such as UniGene Clusters) or genornic sequences, from various public or private databases, such as Genbank, UniGene, etc. are cleaned 501. For a review of genetic databases, see, e.g., Searls (2000). Bioinformatics Tools For Whole Genomes. Annu. Rev. Genom. Hum. Genet. 1: 251-279, which is incorporated herein by reference for all purposes. A catalog of genetic databases is available at http://www.ebi.ac.uk/biocat/, last visited on Jan. 11, 2000, the content of the web site is incorporated herein by reference for all purposes.

Cleaning sequences may be as late as into the probe design phase in at least some embodiments. However, in preferred embodiments, the cleaning is performed as early as possible, i.e., in the first stage of the entire sequence selection phase, particularly if UniGene sequences (http://www.ncbi.nlm.nih.gov) are used as input to the sequence selection. The output of the cleaning process 501 is a set of cleaned EST/mRNA sequences. Cleaning is often necessary because, even though UniGene had screened its sequences against ribosomal RNAs, vector contamination, and low-complexity regions, a large numbers of UniGene sequences with repetitive elements, low complexity regions, and ambiguous regions still exist.

Cleaning sequences early offers several advantages. Poor quality sequences may interfere with sequence clustering and alignment tools' capability to refine clusters and align assemblies. For example, the following sequence has a high low-complexity region (underlined region):

attccgggttagcctgaccgcgcgcgcgcgcgcgcgcg

Another advantage for cleaning raw sequences early is to prevent poor sequence regions from being considered for probe picking. In the following example sequence segment, a region in which the actual probes (25 base long) would be picked is underlined:

gatcgattccgattccgggttagcctgaccgaaaaaaaaaaaaaaa

Obviously, if runs of A's are detected and masked out, the probe would not have been selected (the 3′-end of the probe would have been . . . ccgnnn, with 3 ambiguous bases). Therefore, by eliminating low quality regions from the design sequence, probes are less likely to be selected at the poor quality regions. The poor quality probe may not be eliminated during the probe selection phase, since probe sequences are shorter, and, therefore, may not present enough clues of artifacts.

In a preferred embodiment, the UniGene sequences are cleaned using procedures in the following order:
1) Reduce large EST Cluster size. All the ESTs from large UniGene clusters, which contain more than 500 sequence members are eliminated. This pre-processing is to facilitate clustering.
2) Remove withdrawn sequences. A withdrawn sequence is a sequence without a GI number in the original GenBank description.
3) Screen, filter, and mask raw sequences. All sequences may be screened and filtered against vector, repetitive element, mitochondria, ribosomal RNA databases. The filtered regions may be masked using the ambiguous base code, N.
4) Trim terminal ambiguous sequence regions. the sequence regions from the end of raw sequences are trimmed if the region contains a high proportion of ambiguous bases. An ambiguous region is defined as having at least one ambiguous base, N, for each 10-base window from a sequence end.

In a particularly preferred embodiment, a score, Nₚᵣ, is calculated as the maximal number of good probes selectable in a given DNA strand, and used it as a measure of sequence quality. In some embodiments, this score is calculated for each sequence before and after each stage of processing. In preferred embodiments, however, this score may only be calculated for candidate consensus sequences.

Once the raw sequences are cleaned, they are used to form clusters. Clustering may be performed using any suitable clustering tools, such as the Pangea's EST clustering and alignment tools (CAT, DoubleTwist, Oakland, Calif.), see, also, Burke et al., 1999, d2_cluster: A Validated Method for Clustering EST and Full-Length cDNA Sequences, Genome Research 9:1135-1142, incorporated herein by reference in its entirety for all purposes.

In preferred embodiments, the clusters are based upon existing cluster structures such as the UniGene cluster structure. In one embodiment, reclustering of UniGene, i.e., subject all the sequences from an existing cluster structure to de novo clustering, may be used. Re-clustering can correct the heterogeneity problem of the UniGene clusters.

In a particularly preferred embodiment, UniGene clusters are sub-clustered to make it more suitable for probe selection while maintaining the original supercluster structures 502. Subclustering, as used herein, refers to a process of re-clustering sequences within the same raw cluster, such a UniGene Cluster, using more stringent clustering criteria.

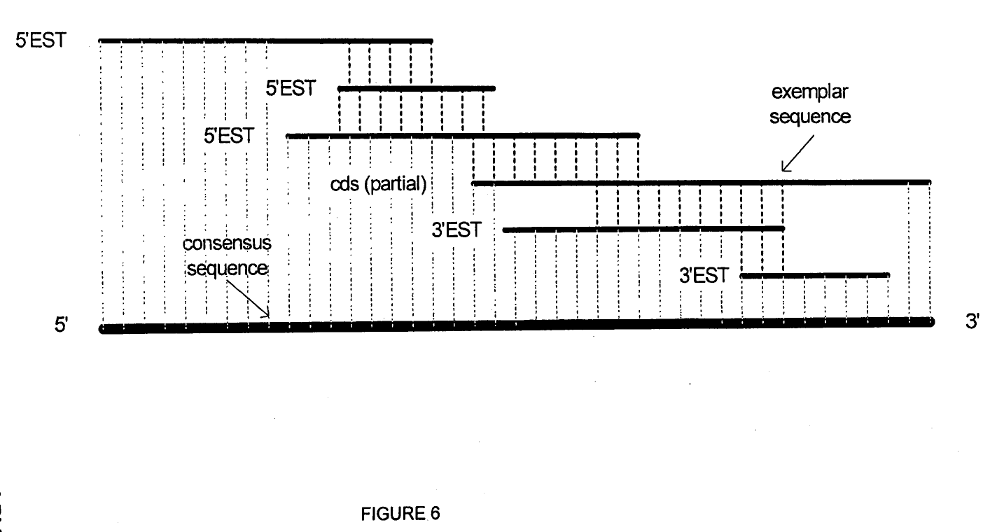

Continuing the process shown in FIG. 5, the subclusters are used to generate consensus or exemplar sequences 503. Sequence assembly is generated, preferably, for each subcluster, using a contig assembly tool or a multiple sequence alignment tool, such as the one provided by the CAT. Sequence alignment, as used herein, refers to a collection of subcluster sequences that are aligned with one another. These sequences, or “sequences in assembly”, often connect to one another, sharing similarity at the sequence ends or in the center as illustrated in FIG. 6. Note that multiple thin horizontal lines are used to represent assembled sequences (including the exemplar sequence) and a bold horizontal line to represent the consensus sequence.

An “exemplar sequence”, or an “exemplar”, as used herein, refers to an original sequence (the fourth sequence in FIG. 6) in a subcluster sequence assembly representative of the entire subcluster assembly. A “consensus sequence” (the last sequence as a bold line in FIG. 6), or a “consensus”, as used herein, refers to a “virtual sequence”, with its each base pooled from each corresponding base position of the subcluster assembled sequences. Either a consensus sequence or an exemplar sequence can be used as a candidate sequence.

In some embodiments, an exemplar sequence is used to represent a subcluster sequence assembly. The advantages of using an exemplar sequence include:
1) An exemplar sequence is a natural sequence, and therefore has biological significance.
2) The quality of an exemplar sequence does not decrease when the number of assembled sequences in the subcluster increase.
3) There is less chance of getting a chimeric clone for exemplar sequences as opposed to using consensus sequence.
4) Exemplar sequence description may be used directly, resulting in computational cost saving.

In a particularly preferred embodiment, the exemplar sequence of a subcluster is determined by using the short form sequence alignment information within that subcluster. When picking exemplars, two criteria may be applied in the following order of priority:
1) Sequence Type. The order of preference is: cds sequences >5′ESTs>3′ESTs> directionless ESTs.
2) Sequence Quality. A high preference is given to sequences with the maximal number (at least 500 bp) of non-ambiguous bases spanning across the consensus sequence's 3′ end.

In FIG. 6, for example, the fourth sequence is chosen as an exemplar, because it is a cds sequence aligned to the consensus sequence's 3′ end for over 500 bp (length scale not shown in the figure). Some of the advantages of the exemplar sequences include its sensitivity to sequencing errors. A few sequencing errors in the 3′ end of the exemplar sequences will adversely affect the expression probe array performance, if probes happens to be selected in the region with errors.

One advantage of using consensus sequences for chip design is that they result in overall improved sequence quality. Most sequences (and, particularly EST sequences) that are available in the public databases have an average error rate of approximately 6%. The errors include sequencing errors, frame-shifts, mislabeling, clone reversals (reverse complement 3′ EST clones), and chimeric clones. By using a consensus sequence from each cluster of highly similar sequences, these errors may be minimized, and the consensus sequences may provide higher-quality sequences for probe design.

In preferred embodiments, the composition of each consensus sequence is determined using consensus base-calling rules. Different types of bases for each aligned position may be given different weight. Table 1 shows an exemplary weight matrix. In the matrix, a unit weight of 1 is assigned to all the regular bases (A, T or U, G, C). An ambiguous base (N), or any base otherwise (X), carries a weight of 0.5. An external gap (.) is defined as a gap lying within 10 contiguous regular bases from either the 5′-end or the 3′-end of the sequence being aligned. Otherwise, the gap is an internal gap. Once an external gap is found at the sequence end, all the 10 contiguous regular bases at sequence end are filtered and masked to ambiguous bases, N's, and all the external gaps is assigned a weight of 0. For internal gaps, their bases are assigned a weight of 1, same as regular bases.

TABLE 1
The weight matrix for consensus sequence calls

| Code | A | T/U | G | C | N/X | .(internal gap) | .(external gap) |
|---|---|---|---|---|---|---|---|
| Weight | 1 | 1 | 1 | 1 | 0.5 | 1 | 0 |
| (W) | | | | | | | |

A vector Vᵢ={N_{A}, N_{T}, N_{G}, N_{C}, N_{N}, N_{gap}}ᵢ is computed for each position i of the entire consensus sequence. The values of N_{A}, N_{T}, N_{G}, N_{C}, N_{N}, N_{gap} may be defined as the following, where n is the total number of sequences in the aligned assembly:

| | |
|---|---|
| N_{A} = | Sum of appearance of A's * W_{A}/n |
| N_{T} = | Sum of appearance of T's and U's * W_{T/U}/n |
| N_{G} = | Sum of appearance of G's * W_{G}/n |
| N_{C} = | Sum of appearance of C's * W_{C}/n |
| N_{N} = | Sum of appearance of N and X's * W_{N/X}/n |
| N_{gap} = | Sum of appearance of internal gaps * W_{internal gap}/n |

Then, a “75% rule” may be used to generate the consensus base for position i, based on the computed vector Vi. The rule allows the assignment of a base if its weighted appearance for that position is greater than 0.75:

| | | |
|---|---|---|
| Base(i) = | A | when N_{A} > 0.75, |
| | T | when N_{T} > 0.75, |
| | G | when N_{G} > 0.75, |
| | C | when N_{C} > 0.75, |
| | . | when N_{gap} > 0.75, |
| | N | when N_{N} > 0.75, |
| | N | when N_{A} < 0.75 and N_{T} < 0.75 |
| | | and N_{G} < 0.75 and |
| | | N_{C} < 0.75 and N_{gap} < 0.75. |

The consensus bases may be concatenated together; terminal gap bases are removed, and the consensus sequence is outputted.

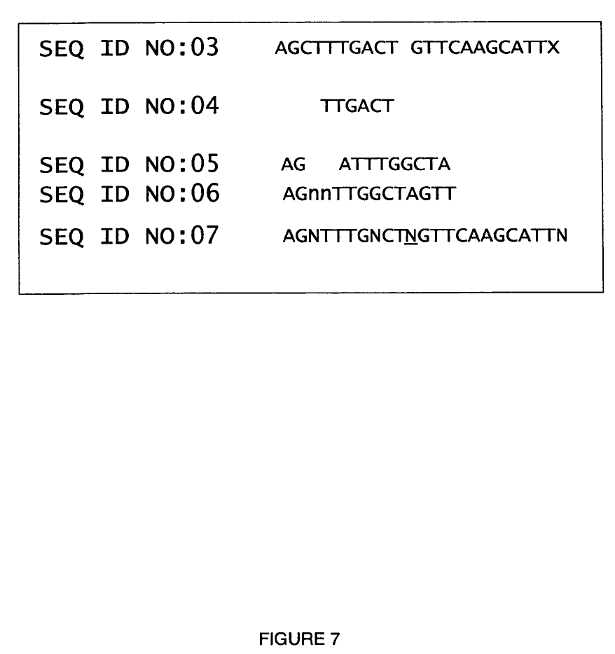

As an example, FIG. 7 shows the determination of consensus sequence X from raw sequences A, B, C, D.

In another preferred embodiment, quality scores are derived from the trace file of sequence data (e.g., ABI sequencer trace). The quality scores may be used to improve the quality of consensus sequences. The quality score may give a measurement of the quality of each base. A higher weight may be given to a high quality score of a base.

In one aspect of the invention, methods, systems and computer software are provided to determine the direction of each individual consensus sequence using the description from its underlying assembly sequences and the sequence alignment information in the short form. The methods, systems and computer software are not only useful for sequence selection for probe array design, but also generally useful for gene-indexing projects

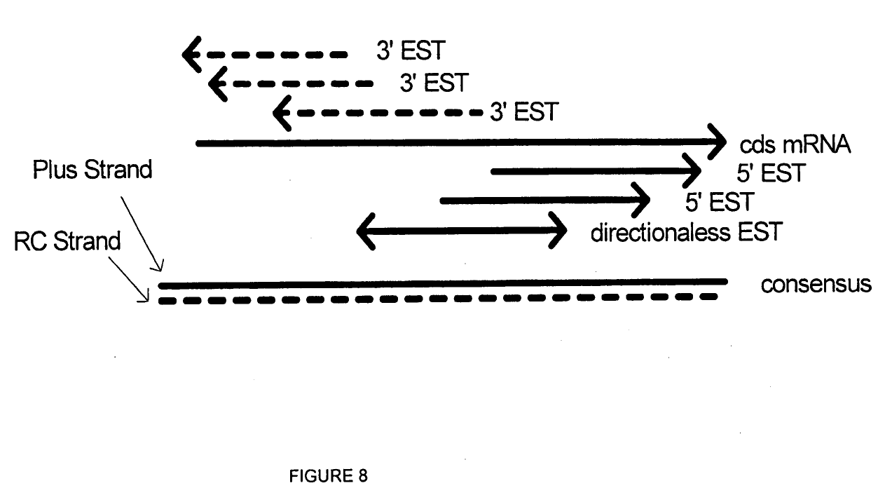

FIG. 8 illustrates an assembled subcluster with all its sequence members, and a consensus sequence. Solid lines indicate a sequence in the same strand as the original sequence before alignment (or, matching the plus strand of the consensus sequence). Dotted lines indicate a sequence in the reverse complement (RC) strand of the original sequence before alignment (or, matching the RC strand of the consensus sequence). Line arrows are drawn to indicate the type of sequences according to their original descriptions. A line with a single arrow pointing left indicates 3′ESTs, a line with a single arrow pointing right indicates 5′ESTs or cds mRNAs, and a line with two arrows pointing in both directions indicates directionless ESTs.

In this figure, it is shown that the reverse complements of three 3′ESTs aligns with two 5′ESTs and one cds mRNAs. As an alternative to the above graphical notation, a mathematical notation system may be used for ease of description. A (s, d) pair may be used to record both the sequence direction digested from the original sequence description, s, and the strand information derived from subcluster alignment, d. The variable s can take the value of 5 (for 5′EST or cds mRNA), 3 (for 3′EST), 0 (for directionless EST), or con (for consensus sequence). The strand variable d can take the value of + (for Plus Strand) or − (for RC Strand). Possible combination values of (s,d) pair are summarized in Table 2. Using this notation, the total number of sequences of type s which align with the consensus strand d, N_{s,d} may be easily calculated. For the example in FIG. 8, N_{5,+}=3, N_{3,-}=3, and N_{0,+}=1.

TABLE 2
All possible values of the pair, (s, d), for different sequences.

| | Sequence Type | | | |
|---|---|---|---|---|
| Strands in | | 5′ EST and | Directionless | Consensus |
| Assembly | 3′ EST | cds mRNA | EST | sequence |
| Plus Strand | (3, +) | (5, +) | (0, +) | (con, +) |
| R.C. Strand | (3, −) | (5, −) | (0, −) | (con, −) |

In general, it may be assumed that each 3′EST should align with the reverse complement strand (RC Strand) of whichever strand all 5′ESTs and cds mRNAs align with. This assumption enables easy deduction of the direction of consensus sequences solely based on the (s,d) pairs for all the sequences in the subcluster assembly. The assumption generally holds true, because the majority of sequencing projects deposit 3′ EST as they are originally sequenced, without going to the lengths of reverse complementing them. However, in some instances, this assumption is not true and methods are provided to resolve conflicts.

In some preferred embodiments, the following relationships are used throughout the analysis:
1) Any 5′ EST or cds gene aligning with the plus strand of the consensus sequence, and any 3′ EST or cds gene aligning with the RC strand of the consensus sequence are regarded as consistent with the plus strand of the consensus sequence.
2) Any 5′ EST or cds gene aligning with the RC strand of the consensus sequence, and any 3′ EST or cds gene aligning with the plus strand of the consensus sequence are regarded as consistent with the RC strand of the consensus sequence.
3) Any directionless EST, regardless what strand it aligns with of the consensus sequence, is regarded as consistent with either the plus strand or the RC strand of the consensus sequence.

Below, the three basic relationships restated using formulas:
N_{con,+}=N_{5,+}+N_{3,-}+N_{0,+}
N_{con,-}=N_{5,-}+N_{3,+}+N_{0,-}

The above formula may be used to calculate the number of assembly sequences consistent with the plus strand of the consensus N_{con,+}, and the number of assembly sequences consistent with the RC strand of the consensus N_{con,-}. Applying the above two formulas to the example in FIG. 8, it can be concluded that all the sequences in the example are consistent with each other. Therefore:
N_{con,+}=N_{5,+}+N_{3,-}+N₀ =7
Because N_{con,+}is equal to the number of all the participating sequences in the subcluster, it can be concluded that the consensus sequence in plus strand has a direction consistent with 5′ESTs and cds mRNAs. Therefore, the direction label of ‘5’ is assigned to the consensus sequence (d=‘5’).

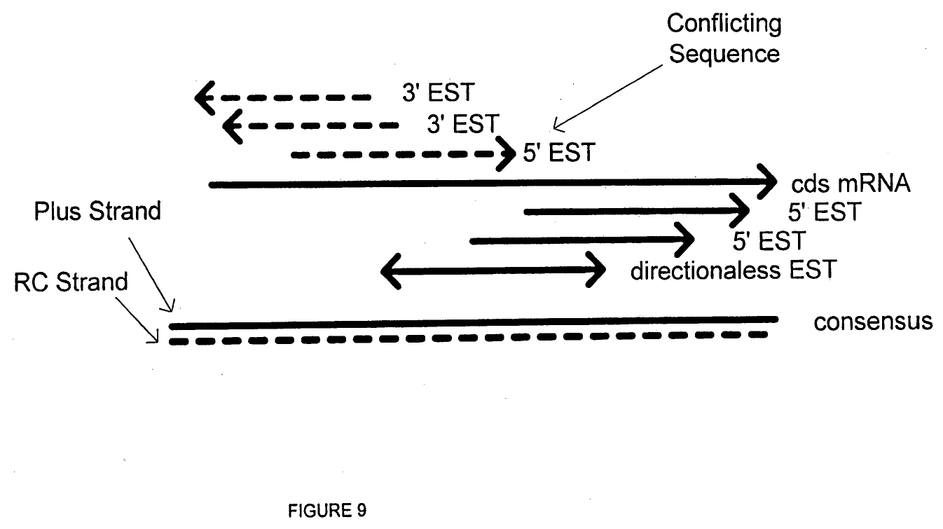

Inconsistent sequence description problem can arise for many reasons. FIG. 9 shows an example of the problem. For this example, the number of sequences consistent with the plus strand, and the number of assembly sequences consistent with the RC strand can be calculated, respectively:
N_{con,+}=N_{5,+}+N_{3,-}+N_{0,+}=6
N_{con,-}=N_{5,-}+N_{3,+}+N_{0,-}=1
The majority consensus sequence direction, Dₘₐⱼₒᵣ, and the minority consensus sequence direction, Dₘᵢₙₒᵣ can be calculated using the following formula:
Dₘₐⱼₒᵣ=5′, when N_{con,+}>N_{con,-}
3′, when N_{con,-}>N_{con,+}
Dₘᵢₙₒᵣ=3′, when N_{con,+}>N_{con,-}and N_{con,-}>0
5′, when N_{con,-}>N_{con,+} and N_{con,+}>0
NULL, when N_{con,-}=0 or N_{con,+}=0
For the example in FIG. 9, Dₘₐⱼₒᵣ=5′ and Dₘᵢₙₒᵣ=3′. Since N_{con+}*N_{con-}<>0 (or, Dₘᵢₙₒᵣ<>NULL), the assembly sequence direction labels are inconsistent with the alignment results, i.e., it can not be determined with certainty that the consensus sequence is in 5′ direction, because there is one 5′EST inconsistent with the plus stand of the consensus. Exact reason for such inconsistency is often unknown. Possible reasons include:
1) Mislabeling. 5′ EST clones may be mistakenly labeled as 3′EST clones by error, and vice versa.
2) Clone reversal.
3) Chimeric clones. The result is that the wrong assembly serves as the centerpiece of the subcluster, sharing similarity with one group of sequences at 5′-end, and sharing similarity with another group of sequences at 3′-end. The two groups of sequences may be totally unrelated.

In one aspect of the invention, methods, systems and computer software are provided to resolve the directional conflicts. In preferred embodiments, when there are contradictory directions in a cluster, the method include determining the probability (b) that the contradictions are explained by random errors according to a statistical model and the weighted number of contradictory sequences in the cluster; and defining the direction of majority of the sequences as the direction of the consensus sequence if the probability is the same as or greater than a threshold value (T).

In one particularly preferred embodiment, the statistical model is a binomial distribution model as follows: in a population of subclusters each with a size of n, for each subcluster, x number of sequences is observed to appear in the minority consensus sequence direction Dₘᵢₙₒᵣ, and n-x number of sequences appearing in the majority consensus sequence direction Dₘₐⱼₒᵣ (assume x<n/2 and x>=0). Assume all sequences in direction Dₘᵢₙₒᵣ occur due to random errors, and the probability of such random error to occur on each sequence is a constant, p, the probability density function is as follows:

$$b ( x ; n , p ) = \frac{n !}{x ! ( n - x ) !} {{p}^{x} ( 1 - p )}^{n - x}$$

First, it is assumed that the probability of random error constant p is small. p is mainly due to mislabeling and clone-reversal errors, which, according to the literature, are at most 5-6%.

For a subcluster size of 7 or greater, the binomial probability is about 0.02%, much less than the probability of wrong labeling (6%). Therefore, one important consequence of this assumption is that Dₘₐⱼₒᵣ can be used interchangeably with the direction of consensus sequence, D_{con}, for subcluster size of 7 or greater.

Second, it is assumed an contradiction of N_{con,+} and N_{con,-}, or, in other words, the inconsistency of Dₘₐⱼₒᵣ and Dₘᵢₙₒᵣ, is caused solely by random errors. Generally, contradiction in sequence labels results from three sources: mislabeling, clone reversal, and chimeric clones. Of the three error sources, sources of random errors include mislabeling of sequences, and clone reversal errors for a small percentage of sequences in the subcluster assembly. This assumption can be relaxed to include system errors, assuming the system errors occurring at a probability significantly lower than the probability of random errors.

The sources of system errors include chimeric clone errors, and clone reversal errors for a large percentage of sequences in the subcluster assembly.

In preferred embodiments, a simple computational method is used to derive p. The method includes obtaining binomial frequency distribution of subclusters of size n over the number of contradictory sequences consistent with Dₘᵢₙₒᵣ. P may be estimated using p′=f¹fₘₐₓ(x)/n, where f¹fₘₐₓ(x) is the value of x when f(x), or b(x; n, p), reaches its maximum.

In more preferred embodiments, a less biased and more practical method is used to estimate p. The above method may be used to analyze many different subclusters with varying size of n₁, n₂, . . . , nₖ. A series of k charts, each showing the binomial frequency distribution of subcluster of size nᵢ(1<=i<=k) over the number of contradictory sequences consistent with D_{minor,I}, may be plotted. f¹fₘₐₓ(xᵢ)/nᵢ may be used to estimate p, where f¹fₘₐₓ(xᵢ) is the value of xᵢ when f(xᵢ), or b(xᵢ; nᵢ, p), reaches its maximum. The estimate p′ of p as: p′=(Σ_{i=1→k}f¹fₘₐₓ(xᵢ)/nᵢ)/k

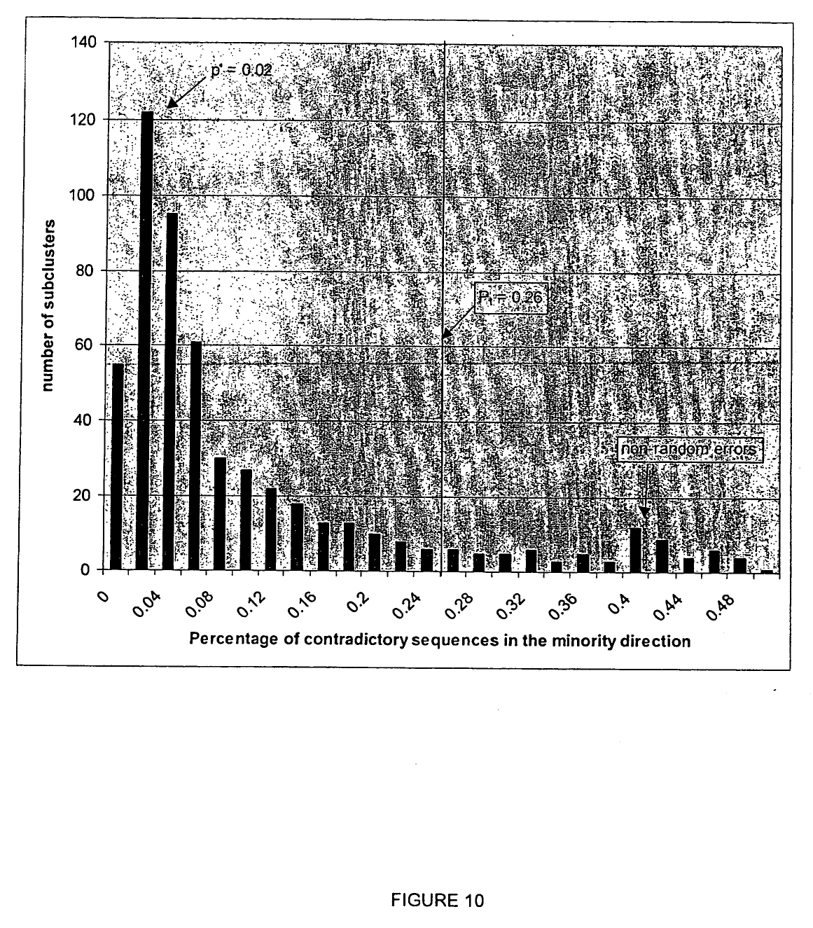

In preferred embodiments, the series of k charts are normalized. The normalization is preferably achieved by transforming the x-axis to take the value r=x/n, a ratio of the number of contradictory sequences in direction Dₘᵢₙₒᵣ over the total number of sequences within the same subcluster assembly. Then, assuming a small variance among different estimated p′ᵢ, a single chart is obtained, showing the additive frequency distribution of subcluster varying in size n₁, n₂, . . . , nₖ (where nᵢ, n₂, . . . , nₖ are consecutive natural numbers) over the ratio r. The equation f¹fₘₐₓ(r) may be used to estimate p, where f¹fₘₐₓ(r) is the value of r=xᵢ/nᵢ when f(r), or Σ_{k=1→i}b(xₖ; nₖ, p), reaches its maximum. FIG. 10 shows that the estimate p′= [symbol] f¹fₘₐₓ(r)=0.02.

The second assumption mentioned earlier may be relaxed to include non-random errors. In FIG. 10, a fairly large number of subclusters are observed with a large percentage of contradictory sequence in direction Dₘᵢₙₒᵣ. As discussed earlier, these “non-random errors” occur systematically, mainly due to chimeric clone errors, and clone reversal errors for a large percentage of sequences in the subcluster assembly. A close investigation of about ten such subclusters confirmed the existence of chimeric clones.

A binomial cutoff threshold (T) is derived to separate system errors from random errors. If the probability is above T, the contradictions are explainable by random errors. In preferred embodiments, the threshold T=0.002 in combination with a p=0.06. Alternatively, a threshold value of x/n, Pₜ, may be used to estimate the breakpoint above which system errors dominate over random errors. For example, if Pₜ=0.26, as shown in FIG. 10, all contradiction x in the x>n*0.26 range may be due to system errors.

Having obtained the estimates of p, and T or Pₜ, the entire consensus sequence direction resolution rules can be deduced as tabulated in Table 3. It is preferable to give weights to different types of sequence in calculating n and x. In a preferred embodiment, the following weights are given to different types of sequences: cds/mRNA carrys a weight of 10, 5′EST carrys a weight of 1, 3′EST carrys a weight of 1, directionless EST carrys a weight of 0.

Note in the table that for significant contradictions that are not explainable by random errors, the consensus sequence is labeled as a tentative ‘?’, and subcluster both groups, group Dₘₐⱼₒᵣ and Dₘᵢₙₒᵣ, again to eliminate the contradiction.

TABLE 3
Summary Of The Consensus Direction Resolution Algorithm.

| Subcluster Status | Detection Criteria | D_{con} Label |
|---|---|---|
| All member sequences | n = N_{0,+} + N_{0,-} | ‘?’ |
| are direction-less ESTs. | | |
| All sequence descriptions | x = 0 | Dₘₐⱼₒᵣ |
| fits well with alignment | | |
| results. | | |
| Significant contradictions | b(x; n, p′) < T and x > n * p′ | ‘?’ (subject to |
| that are not explained by | (or: estimate using x > n * Pₜ) | further sub- |
| random errors. | | cluster for each |
| | | group Dₘₐⱼₒᵣ |
| | | and Dₘᵢₙₒᵣ) |
| Contradictions that can | (1) b(x; n, p′) ≧ T and | Dₘₐⱼₒₓ |
| be explained by random | x ≠ n/2; or, (2) b(x; n, p′) < T | |
| errors. | and x ≦ n * Pₜ | |
| | (or: estimate using x < n * Pₜ₎ | |
| A tie between Dₘₐⱼₒᵣ and | b(x; n, p′) ≧ T and x = n/2 | ‘?’ |
| Dₘᵢₙₒᵣ | | |
| (i.e., N_{con,+} = N_{con,-}) | | |
| exists. | | |
| (n is the total number of sequences in a subcluster. x is the number of contradictory sequences in the direction Dₘᵢₙₒᵣ. p′ is the probability of random errors of the sample. T is the binomial probability threshold above which random errors are dominant over non-random errors. | | |
| # Pₜ is the x/n value threshold above which non-random errors dominate over random-errors.) | | |

Referring to the process in FIG. 5, the final selection step 505 is picking final design sequences. In preferred embodiments, probe design sequences are selected based on three factors: subcluster composition, the relative size of subclusters, and sequence quality (Qₐ) which is a measure of consensus sequence quality using the following formula:

$$\begin{matrix}{Q}_{a} = ( {N}_{Pr} ( {D}_{con} ) - 50 ) * {( {N}_{sub \text{-} seq} )}^{1 / 2} , & ( {N}_{Pr} ( {D}_{con} ) >= 50 ) \\ = ( {N}_{Pr} ( {D}_{con} ) - 20 ) * {( {N}_{sub \text{-} seq} )}^{1 / 2} * 0.01 \% , & ( 50 > {N}_{Pr} ( {D}_{con} ) >= 20 ) \\ = 0 , & ( {N}_{Pr} ( {D}_{con} ) < 20 )\end{matrix}$$

Where Nₚᵣ(D_{con}) is the number of good probes available from the 3′-end of the consensus sequence (as dependent on consensus direction D_{con}) and N_{sub-seq} is the number of non-discarded sequences in the subcluster. Note that when consensus sequence direction is ambiguous (labeled as ‘?’), Nₚᵣ=max(Nₚᵣ(5′), Nₚᵣ(3′)).

One exemplary sequence picking logic is summarized in Table 4. Each row contains a subcluster composition property, a subcluster relative size property, and, if both properties hold, the subcluster to pick.

TABLE 4
Sequence Picking Logic.
T % is a threshold value. In a particularly preferred embodiment,
T % = 70%.

| Property of Subclusters (within the same | | |
|---|---|---|
| supercluster) | | |
| Subcluster | | |
| Composition | Subcluster Relative Size | Subcluster to Pick |
| Not Exists: any non- | Exists: 1 EST subcluster | The largest |
| EST subclusters | size > T % supercluster size | subcluster |
| Not Exists: any non- | Not Exists: any EST | Top 2 largest |
| EST subclusters | subcluster size > T % | subclusters |
| | supercluster size | |
| Exists: >= 1 non- | Exists: 1 non-EST subcluster | The largest non-EST |
| EST subcluster | size > T % supercluster size | subcluster |
| Exists: = 1 non- | Not Exists: any non-EST | The largest non-EST |
| EST subcluster | subcluster size > T % | subcluster and the |
| | supercluster size | non-EST subcluster |
| Exists: >= 2 non- | Not Exists: any non-EST | Top 2 largest |
| EST subcluster | subcluster size > T % | non-EST subclusters |
| | supercluster size | |

In preferred chip sequence picking rules, compatibility rules may be used to either include or exclude sequences from previous chip designs. One or more old sequence sets (for previous chip design) and a new candidate clustered sequence set may be inputted. Compatibility between each old sequence set and the new candidate sequence set may be determined. One exemplary process includes sequence matching between sequences in the old sequence set and sequences in the new candidate sequence set. Sequence match may be performed as a pair of sequences, one from each set, having some common properties, such as same GenBank accession numbers or highly similar sequence contents. Table 5 shows exemplary matching criteria for designing a probe array that is compatible with an early version of the probe array.

TABLE 5
Sequence matching criteria for backwards compatibility

| Criterion Code | Criterion Description |
|---|---|
| A | Sequence pair from two matching sets matches both the |
| | GenBank accession numbers and the sequence direction; |
| | and, matching sequence in the new candidate sequence |
| | set must align within 100 bp of the 3′-end of its |
| | subcluster consensus sequence |
| B | Each sequence in the old sequence set is not duplicated in |
| | the new candidate sequence set in terms of the GenBank |
| | accession number. |
| C | Each subcluster involved should consist of only EST |
| | sequences. |

The backwards compatible design process also includes compatible match between the matched sequence in the old sequence set and the subcluster in which the corresponding matched candidate sequence was discovered in the new candidate sequence set. In preferred embodiments, compatible match as matching takes place at the subcluster level. However, a compatible matching may also take place at the sequence level, or at the supercluster level.

The backwards compatible design process further includes excluding, including, or replacing the matched subclusters in the new candidate sequence set for the sequence picking process, depending on the type of old sequence set involved. In preferred embodiments, all the matching subclusters and their relationship with old sequences for subsequent selection of probe need to be tracked, regardless of whether they end up in the final design or not. An old sequence list can be divided into three basic types:
1) Exclusion Set (Sₑ). Compatibly matched subclusters of each sequence in this set, Sₑ, are excluded from being picked for the final design. Note that this exclusion is only meaningful during the sequence selection phase. In the probe selection phase, the pre-designed probe sets may be added back.
2) Inclusion Set (Sᵢ). Compatibly matched subclusters of each sequence in this set, Sᵢ, are included into the final design.
3) Replacement Set (Sᵣ). Compatibly matched subclusters of each sequence in this set, Sᵣ, are tracked even though the matched subclusters do not get any preference to include into or exclude from the final design.

### CONCLUSION

The present invention provides methods, systems and computer software products for nucleic acid probe array design. It is to be understood that the above description is intended to be illustrative and not restrictive. Many variations of the invention will be apparent to those of skill in the art upon reviewing the above description. The scope of the invention should not be limited with reference to the above description, but should instead be determined with reference to the appended claims, along with the full scope of equivalents to which such claims are entitled.

All cited references, including patent and non-patent literature, are incorporated herein by reference in their entireties for all purposes.

1-12. (canceled)

13. A method of obtaining candidate sequences for designing a probe array comprising:
cleaning raw sequences;
refining clusters of the raw sequences;
generating candidate design sequences, wherein the candidate design sequences are exemplar or consensus sequences of the clusters; and
selecting probes targeting the exemplar or consensus sequences for designing the probe array.

14. The method of claim 13 wherein the cleaning comprises removing withdrawn sequences;
screening and filtering and masking raw sequences; and
trimming terminal ambiguous sequence regions.

15. The method of claim 13 wherein the refining includes two level clustering.

16. The method of claim 13 wherein the generating comprises:
selecting exemplary sequences.

17. The method of claim 16 wherein the generating comprises:
generating alignments of sequences within clusters;
calling consensus sequence bases according to consensus calling rules; and
determining consensus sequence direction.

18. The method of claim 17 wherein the determining comprises defining the direction of sequences in the clusters as the consensus sequence direction if there is no contradictory sequence directions.

19. The method of claim 18 wherein the determining further comprises
determining the probability (b) that the contradictions are explained by random errors according to a statistical model and the weighted number of contradictory sequences in the cluster; and
defining the direction of majority of the sequences as the direction of the consensus sequence if the probability is the same as or greater than a threshold value (T) and x≠n/2, wherein x is the number of the contradictory sequences and n is the weighted number of the sequences in the cluster.

20. The method of claim 19 wherein the statistical model is a binomial distribution and the probability is calculated as follows:
$$b ( x ; n , p ) = \frac{n !}{x ! ( n - x ) !} {{p}^{x} ( 1 - p )}^{n - x}$$
wherein n is the weighted number of the sequences in the cluster; p is the probability of random errors resulting in the contradictions; and x is the number of the contradictory sequences.

21. The method of claim 20 wherein coding regions CDS and mRNA sequences carries a higher weight than 5′EST or 3′EST; directionless EST carrys a weight of 0.

22. The method of claim 21 wherein the weights to different types of sequences are the same.

23. The method of claim 22 wherein the threshold value is around 0.001.

24. The method of claim 22 wherein the threshold value is around 0.002.

25. The method of claim 22 wherein the threshold value is around 0.003.

26. The method of claim 22 further comprising defining the direction of majority of the sequences as the direction of the consensus sequence if the probability is lower than the threshold value and x≦n*(Pₜ), wherein Pₜ is a threshold value of x/n above which non-random errors dominate over random errors.

27. The method of claim 26 further comprising further subclustering for the minority direction and majority direction if the probability is smaller than the threshold value and x>n*Pₜ.

28. The method of claim 27 wherein the p is between 0.03-0.10.

29. The method of claim 27 wherein the p is around 0.06.

30. The method of claim 27 wherein the p is determined according to binomial frequency distribution of contradictory sequences in a plurality of clusters or subclusters of sequences.


SEQUENCE LISTING (7 sequences)

SEQ ID NO: 1 - DNA, 38 bp, Homo Sapien
attccgggttagcctgaccgcgcgcgcgcgcgcgcgcg

SEQ ID NO: 2 - DNA, 46 bp, Homo Sapien
gatcgattccgattccgggttagcctgaccgaaaaaaaaaaaaaaa

SEQ ID NO: 3 - DNA, 21 bp, Homo Sapien
agctttgactgttcaagcatt

SEQ ID NO: 4 - DNA, 6 bp, Homo Sapien
ttgact

SEQ ID NO: 5 - DNA, 11 bp, Homo Sapien
agatttggcta

SEQ ID NO: 6 - DNA, 14 bp, Homo Sapien
  misc_feature (3)..(4): n is a, c, g, or t
agnnttggctagtt

SEQ ID NO: 7 - DNA, 23 bp, Homo Sapien
  misc_feature (3)..(3): n is a, c, g, or t
agntttgnctngttcaagcattn